(19) United States
(12) Patent Application Publication
(10) Pub. No.: US 2021/0085636 A1
(43) Pub. Date: Mar. 25, 2021

(54) THERAPEUTIC AGENT FOR GLAUCOMA COMPRISING FP AGONIST AND ß BLOCKER

(71) Applicant: ONO PHARMACEUTICAL CO., LTD., Osaka (JP)

(72) Inventors: Shinsaku YAMANE, Osaka (JP); Satoshi NAKAYAMA, Osaka (JP); Kazufumi NAGAI, Osaka (JP); Kazumi MORIYUKI, Osaka (JP); Tomohiro KARAKAWA, Osaka (JP)

(73) Assignee: ONO PHARMACEUTICAL CO., LTD., Osaka (JP)

(21) Appl. No.: 16/954,416

(22) PCT Filed: Dec. 20, 2018
(86) PCT No.: PCT/JP2018/047102

§ 371 Date: Jun. 16, 2020

(30) Foreign Application Priority Data
Dec. 21, 2017 (JP) 2017-244573

(51) Int. Cl. A61K 31/335; A61K 31/5377; A61K 31/4704; A61K 31/138; A61K 31/352; A61K 31/343; A61P 27/06

(52) U.S. Cl. CPC A61K 31/335; A61K 31/5377; A61K 31/4704; A61P 27/06; A61K 31/352; A61K 31/343; A61K 31/138

(57) ABSTRACT

A combination of the present invention is an effective therapy for glaucoma, in particular a combination of 2-propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate and a β-blocker, is useful as an agent for treating glaucoma since the combination enhances intraocular pressure lowering action compared to a single administration of each drug and has an effect of maintaining intraocular pressure lowering action.

### TECHNICAL FIELD

The present invention, in one embodiment, relates to a therapeutic agent for glaucoma comprising an FP agonist as an active ingredient adapted to be administered in combination with a β-blocker, wherein the FP agonist is a compound represented by the following formula:

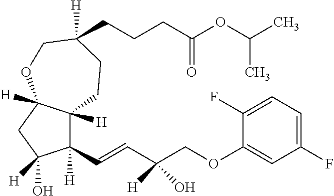 [SMILES: CC(C)OC(=O)CCC[C@H]1CC[C@@H]2[C@@H](/C=C/[C@@H](O)COc3cc(F)ccc3F)[C@H](O)C[C@@H]2OC1]

wherein all symbols have the same meanings as those described later.

### BACKGROUND ART

Glaucoma is an eye disease characterized by disturbance of visual function causing transient or permanent visual field defect and reduced visual acuity. Aqueous humor is accumulated due to disturbance of aqueous humor circulation, and intraocular pressure is continuously increased, resulting in compression of the optic nerve. In the treatment of glaucoma, it is effective to lower intraocular pressure, and for example, drug therapy (eye drops, internal medicine, and infusion treatment), laser therapy, and surgical therapy are performed to lower intraocular pressure.

An FP agonist which is used as an active ingredient in the present invention is known as a medicine useful as an agent for preventing and/or treating eye diseases and the like (see Patent Literature 1). Among others, 2-propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate is known as Sepetaprost (international generic name) (hereinafter sometimes abbreviated as Compound A) and the development as a therapeutic agent for glaucoma is underway.

At the treatment site of glaucoma, it is common to use drugs with different mechanisms of intraocular pressure lowering in combination, and for example, Xalacom (registered trademark) Combination Eye Drops (latanoprost-timolol maleate combination), Tapcom (registered trademark) Combination Eye Drops (tafluprost-timolol maleate combination) and the like are commercially available.

On the other hand, Patent Literature 1 describes Compound A as Example 16 (25), and as an example of other drugs for complementing and/or enhancing preventing and/or treating effects on glaucoma of Compound A and the like, β-blockers are described.

### CITATIONS LIST

Patent Literature 1: WO 2011/013651

### SUMMARY OF INVENTION

### Technical Problems

An object of the present invention is to find effective therapy for glaucoma and to provide as a medicine.

### Solutions to Problems

The present inventors have conducted intensive studies to achieve the object, and as a result, have found that the object can be achieved by a combination of an FP agonist and a β-blocker (hereinafter, sometimes abbreviated as the combination of the present invention).

The present invention provides, for example, the following embodiments. The embodiments include:

[1] an agent for treating glaucoma or ocular hypertension, comprising an FP agonist as an active ingredient adapted to be administered in combination with a β-blocker or a pharmaceutically acceptable salt thereof, wherein the FP agonist is 2-propanyl 4-{(3 S,5aR,6R,7R,8 aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate;

[2] the agent according to the above [1], wherein the β-blocker is timolol, carteolol, levobunolol, betaxolol, nipradilol, or befunolol;

[3] the agent according to the above [1] or [2], wherein the β-blocker is timolol;

[4] the agent according to any one of the above [1] to [3], wherein the β-blocker or a pharmaceutically acceptable salt thereof and 2-propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate are administered separately or concurrently;

[5] a pharmaceutical composition for treating glaucoma or ocular hypertension, comprising 2-propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate and a β-blocker or a pharmaceutically acceptable salt thereof adapted to be administered in combination;

[6] the pharmaceutical composition according to the above [5], wherein the β-blocker is timolol, carteolol, levobunolol, betaxolol, nipradilol, or befunolol;

[7] the pharmaceutical composition according to the above [5] or [6], wherein the β-blocker is timolol;

[8] the pharmaceutical composition according to any one of the above [5] to [7], which is an eye drop or an eye ointment;

[9] the pharmaceutical composition according to any one of the above [5] to [8], which is a compounding agent;

[10] a method for treating glaucoma or ocular hypertension, the method comprising administering in combination to a patient in need of treating glaucoma or ocular hypertension an effective amount of 2-propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate and a β-blocker or a pharmaceutically acceptable salt thereof, separately or concurrently;

[11] a method for treating glaucoma or ocular hypertension, the method comprising administering to a patient in need of treating glaucoma or ocular hypertension an effective amount of 2-propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate, the method further comprising administering an effective amount of a β-blocker or a pharmaceutically acceptable salt thereof;

[12] 2-Propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate that is used for treating glaucoma or ocular hypertension in combination with a β-blocker or a pharmaceutically acceptable salt thereof;

[13] a use of 2-propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate for the manufacture of an agent for treating glaucoma or ocular hypertension adapted to be administered in combination with a β-blocker or a pharmaceutically acceptable salt thereof; and the like.

### Effects of Invention

The combination of the present invention is useful as an agent for treating glaucoma since the combination enhances intraocular pressure lowering action compared to a single administration of each drug and has an effect of maintaining intraocular pressure lowering action.

### DESCRIPTION OF EMBODIMENTS

### (1) FP Agonist

In one embodiment, as an FP agonist used in the combination of the present invention, 2-propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate represented by the following formula described in WO 2011/013651:

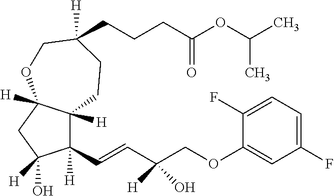 [SMILES: CC(C)OC(=O)CCC[C@H]1CC[C@@H]2[C@@H](/C=C/[C@@H](O)COc3cc(F)ccc3F)[C@H](O)C[C@@H]2OC1]

is mentioned.

Further, as another embodiment of the FP agonist, a compound represented by the general formula (I) described in WO 2011/013651:

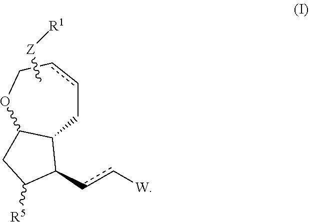

wherein all symbols have the same meaning as those described in WO 2011/013651, a salt thereof, a solvate thereof, or a prodrug thereof is mentioned.

In the present invention, all isomers are included unless otherwise indicated. For example, alkyl groups include straight-chain and branched-chain ones. Furthermore, geometric isomers at double bond, ring, fused ring (E-form, Z-form, cis-form, trans-form), optical isomers due to the presence of asymmetric carbon atom and the like (R and S configurations, α and β configurations, enantiomers, diastereomers), optically active substances having optical activity (D-form, L-form, d-form, l-form), polar substances derived from chromatographic separation (highly polar substances, lowly polar substances), equilibrated compounds, rotamers, mixtures of them in any proportion, and racemic mixtures are all included in the present invention. In addition, all isomers by tautomerism are also included in the present invention.

Further, optical isomers in the present invention may include not only 100% pure ones but also other optical isomers of less than 50% pure.

Compound A can be prepared according to Example 1→Example 2→Example 3→Example 4→Example 5→Example 6→Example 7→Example 8→Example 9→Example 10 (1)→Example 11→Example 12→Example 13→Example 14→Example 15→Example 16 (25) described in WO 2011/013651.

A dose of Compound A used in the combination of the present invention varies depending on age, body weight, symptom, therapeutic effect, administration method, treatment time, and the like. For example, in the case of eye drops, one to a few drops per dropping having a concentration of preferably 0.000001 to 5% (w/v), more preferably 0.00001 to 0.05% (w/v) as a one time amount may be dropped into eyes once to a few times (for example, 1 to 8 times) per day per adult. In addition, in the case of an eye ointment, the eye ointment having a concentration of preferably 0.000001 to 5% (w/w), more preferably 0.00001 to 0.05% (w/w) may be applied once to a few times (for example, 1 to 4 times) per day.

### (2) β-Blocker

In the present invention, a β-blocker is not particularly limited as long as it is a drug that blocks any one of β-adrenergic receptors, that is, each of β1, β2 and β3-receptors among adrenergic receptors of sympathetic nerves, and is used for treating glaucoma. For example, timolol, carteolol, levobunolol, betaxolol, nipradilol, befunolol, or pharmaceutically acceptable salts thereof can be mentioned. Among them, timolol maleate is preferred.

The dose of the β-blocker used in the combination of the present invention varies depending on age, body weight, symptom, therapeutic effect, administration method, treatment time and the like, but a dose obtained a manufacturing approval as a medicine or a lower dose than the dose can be applied. Specific doses for timolol include 0.1 to 0.5% (w/v), preferably 0.25 to 0.5% (w/v). Specific doses for carteolol include 0.1 to 2% (w/v), preferably 1 to 2% (w/v). Specific doses for levobunolol include 0.1 to 0.5% (w/v), preferably 0.25 to 0.5% (w/v). Specific doses for betaxolol include 0.1 to 0.5% (w/v), preferably 0.25 to 0.5% (w/v). Specific doses for nipradilol include 0.1 to 0.25% (w/v), preferably 0.25% (w/v). Specific doses for befunolol include 0.1 to 0.25% (w/v), preferably 0.25% (w/v).

In the present invention, the salt is preferably a pharmaceutically acceptable salt, and is preferably a water-soluble salt. Examples of the pharmaceutically acceptable salt include a salt of an alkali metal (potassium, sodium and the like), a salt of an alkaline earth metal (calcium, magnesium and the like), an ammonium salt, a salt of a pharmaceutically acceptable organic amine (tetramethylammonium, triethylamine, methylamine, dimethylamine, cyclopentylamine, benzylamine, phenethylamine, piperidine, monoethanolamine, diethanolamine, tris(hydroxymethyl)aminomethane, lysine, arginine, N-methyl-D-glucamine and the like), an acid adduct salt (an inorganic acid salt (hydrochloride, hydrobromide, hydroiodide, sulfate, phosphate, nitrate and the like), an organic acid salt (acetate, trifluoroacetate, lactate, tartrate, oxalate, fumarate, maleate, benzoate, citrate, methanesulfonate, ethanesulfonate, benzenesulfonate, toluenesulfonate, isethionate, glucuronate, gluconate and the like) and the like) and the like.

Since all of the above-described β-blockers are commercially available compounds, their toxicity is sufficiently low and they can be used safely as medicines.

Since the combination of the present invention is used for the treatment of glaucoma or ocular hypertension, both the FP agonist and the β-blocker are preferably in dosage forms suitable for the treatment of the diseases, for example, an eye drop, an eye ointment and the like. Each of them may be in a separate dosage form. In addition, the combination of the present invention may be separate preparations, and a compounding agent containing both agents in appropriate amounts, for example, a combination eye drop and a combination eye ointment are also preferable in consideration of medication compliance.

In the combination of the present invention, when the FP agonist and the β-blocker are used in separate preparations, simultaneous administration and administration with a time difference are included. In addition, administration with a time difference means that the FP agonist and the β-blocker are separately administered at a certain time interval. As the order of administration, the FP agonist may be administered first and the β-blocker may be administered later, or the β-blocker may be administered first and the FP agonist may be administered later.

The eye drop and the eye ointment used in the combinations of the present invention can be formulated using commonly used techniques. For example, in the case of eye drops, an isotonizing agent, a buffer agent, a pH-adjusting agent, a solubilizing agent, a thickener, a stabilizer, a preservative, and the like can be appropriately added as additives. In addition, a stable eye drop can be obtained by adding a pH-adjusting agent, a thickener, a dispersing agent and the like and suspending the drug.

### [Toxicity]

The toxicity of the combination of the present invention is sufficiently low and the combination of the present invention can be safely used as a medicine.

### [Application to Medicine]

One embodiment of diseases treated by the combination of the present invention includes glaucoma and ocular hypertension. Examples of glaucoma include acute angle-closure glaucoma, chronic angle-closure glaucoma, secondary angle-closure glaucoma, primary open-angle glaucoma, secondary open-angle glaucoma, congenital glaucoma, normal tension glaucoma, and hypersecretion glaucoma.

In the present invention, the combination of the present invention may be administered in combination with another agent for treating glaucoma for: (1) complementation and/or enhancement of therapeutic effects; (2) improvement of kinetics and absorption, lowering of a dose; and/or (3) alleviation of side effects.

In the present invention, the other agent for treating glaucoma may be any known agent. Examples of the other agent for treating glaucoma include a sympathetic agent (an α2 agonist: for example, apraclonidine hydrochloride and the like, a β2 agonist: for example, dipivefrine hydrochloride and the like), a parasympathomimetic agent (for example, pilocarpine hydrochloride, carbachol, demecarium, echothiophate or distigmine bromide and the like), a sympathetic inhibitor (an al-blocker: for example, bunazosin hydrochloride and the like), a prostaglandin drug (for example, isopropyl unoprostone, latanoprost, bimatoprost, travoprost, an EP2 agonist, an EP4 agonist or a DP agonist and the like), a carbonic anhydrase inhibitor (for example, acetazolamide, diclofenamide, methazolamide, dorzolamide hydrochloride, or brinzolamide and the like), a hyperosmotic agent (for example, glycerin, a combination preparation of glycerin and fructose or isosorbide and the like), a ROCK (Rho-kinase) inhibitor (for example, Y-27632 and the like), and an NMDA antagonist.

### EXAMPLES

Hereinafter, the present invention will be described specifically with reference to Example, but the present invention is not limited thereto.

2-Propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate (Compound A) was used. Compound A can be prepared by a known method. For example, Compound A can be prepared according to Example 1→Example 2→Example 3→Example 4→Example 5→Example 6→Example 7→Example 8→Example 9→Example 10 (1)→Example 11→Example 12→Example 13→Example 14→Example 15→Example 16 (25) described in WO 2011/013651.

### Biological Example 1: Intraocular Pressure Lowering Action

Compound A and a β-blocker were used as test substances. Compound A was prepared in a 3 μg/mL solution using a vehicle (an aqueous solution containing 0.3 w/v % sodium citrate, 0.5 w/v % polysorbate 80, and 0.8 w/v % sodium chloride). Timolol (trade name: Timoptol (registered trademark) 0.5% ophthalmic solution, Santen Pharmaceutical Co., Ltd.) was used as a β-blocker.

Approximately one hour before administering the vehicle and a test substance to male dogs (TOYO Beagle, 16 to 42 months old at start of use) that have been sufficiently acclimated in advance, intraocular pressure in both eyes was measured, and the intraocular pressure was set as the intraocular pressure value before instillation. Thereafter, a test substance or the vehicle was administered by instillation into the treated eyes in an amount of 30 μL each, and intraocular pressure in both eyes was measured about 2, 4, 6, 8, and 24 hours after the administration. The intraocular pressure was measured by a pneumatic applanation tonometer (Model 30 Classic, Reichert, Inc.) under the local anesthesia after instilling 0.4% oxybuprocaine hydrochloride (Benoxil (registered trademark) ophthalmic solution 0.4%, Santen Pharmaceutical Co., Ltd.), which is a local anesthetic. The measurement was performed until a stable value was obtained three times successively (a difference between continuously obtained measured values was within 0.5 mmHg). The average of the three measured values obtained was defined as the intraocular pressure value (mmHg) and was displayed to one digit after the decimal point.

The value obtained by subtracting the intraocular pressure value at each measurement time from the intraocular pressure value before test substance and vehicle administration was defined as the intraocular pressure lowering width, and the average value of the intraocular pressure lowering width of each individual at each measurement time was defined as the average intraocular pressure lowering width. The maximum value of the average intraocular pressure lowering width at each measurement time was defined as the maximum intraocular pressure lowering width. In addition, the average intraocular pressure lowering width 24 hours after administration was defined as the intraocular pressure lowering width 24 hours after administration. The results are shown in Table 1. Meanwhile, in the case where there are two drug names in a group name, it means the order of administration. For example, the Compound A—timolol group means that Compound A was administered first, and timolol was administered thereafter into the same eye.

TABLE 1

| | | | Intraocular pressure | Time after administration |
|---|---|---|---|---|
| | | Maximum intraocular | lowering width 24 hours | exhibiting maximum intraocular |
| | Number | pressure lowering width | after administration | pressure lowering action |
| Group name | of cases | (mmHg ± S.E.) | (mmHg ± S.E.) | (hours) |
| Vehicle | 8 | 0.7 ± 0.48 | 0.4 ± 0.58 | 6 |
| Vehicle - | 8 | 2.7 ± 0.56 | 0.0 ± 0.56 | 2 |
| timolol | | | | |
| Compound A - | 8 | 5.8 ± 0.35 | 3.5 ± 0.75 | 6 |
| vehicle | | | | |
| Compound A - | 8 | 7.0 ± 0.52 | 4.8 ± 0.34 | 8 |
| timolol | | | | |
| Timolol - | 8 | 7.1 ± 0.58 | 4.7 ± 0.57 | 8 |
| Compound A | | | | |

As a result, it was found that the combined use of Compound A and timolol showed a stronger intraocular pressure lowering action compared to each single agent, regardless of the order of administration. Further, it was found that, in the combined use of Compound A and timolol, an intraocular pressure lowering action lasting up to 24 hours after administration was seen, and that there is an effect that the time during which the maximum intraocular pressure lowering action was exerted was longer than that of each single agent.

### INDUSTRIAL APPLICABILITY

The combination of the present invention exerts a significant intraocular pressure lowering action, and is therefore useful for treating glaucoma or ocular hypertension.

1. An agent for treating glaucoma or ocular hypertension, comprising an FP agonist as an active ingredient adapted to be administered in combination with a β-blocker or a pharmaceutically acceptable salt thereof, wherein
the FP agonist is 2-propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate.

2. The agent according to claim 1, wherein the β-blocker is timolol, carteolol, levobunolol, betaxolol, nipradilol, or befunolol.

3. The agent according to claim 1, wherein the β-blocker is timolol.

4. The agent according to claim 1, wherein the β-blocker or a pharmaceutically acceptable salt thereof and 2-propanyl 4-{(3 S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate are administered separately or concurrently.

5. A pharmaceutical composition for treating glaucoma or ocular hypertension, comprising 2-propanyl 4-{(3 S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate and a β-blocker or a pharmaceutically acceptable salt thereof adapted to be administered in combination.

6. The pharmaceutical composition according to claim 5, wherein the β-blocker is timolol, carteolol, levobunolol, betaxolol, nipradilol, or befunolol.

7. The pharmaceutical composition according to claim 5, wherein the β-blocker is timolol.

8. The pharmaceutical composition according to claim 5, which is an eye drop or an eye ointment.

9. The pharmaceutical composition according to claim 5, which is a compounding agent.

10. A method for treating glaucoma or ocular hypertension, the method comprising administering in combination to a patient in need of treating glaucoma or ocular hypertension an effective amount of 2-propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate and a β-blocker or a pharmaceutically acceptable salt thereof, separately or concurrently.

11. A method for treating glaucoma or ocular hypertension, the method comprising administering to a patient in need of treating glaucoma or ocular hypertension an effective amount of 2-propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate, the method further comprising administering an effective amount of a β-blocker or a pharmaceutically acceptable salt thereof.

12. 2-Propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate for treating glaucoma or ocular hypertension in combination with a β-blocker or a pharmaceutically acceptable salt thereof.

13. A method for manufacturing an agent for treating glaucoma or ocular hypertension adapted to be administered in combination, comprising combining 2 propanyl 4-{(3S,5aR,6R,7R,8aS)-6-[(1E,3R)-4-(2,5-difluorophenoxy)-3-hydroxy-1-buten-1-yl]-7-hydroxyoctahydro-2H-cyclopenta[b]oxepin-3-yl}butanoate with a β-blocker or a pharmaceutically acceptable salt thereof.
